(19) United States
(12) Patent Application Publication
(10) Pub. No.: US 2017/0296084 A1
(43) Pub. Date: Oct. 19, 2017

(54) CARDIAC MAPPING CATHETER

(71) Applicant: UNIVERSITY OF UTAH RESEARCH FOUNDATION, Salt Lake City, UT (US)

(72) Inventors: Joshua Jacob Evans BLAUER, East Bountiful, UT (US); Darrell J. SWENSON, San Diego, CA (US)

(21) Appl. No.: 15/510,920

(22) PCT Filed: Sep. 18, 2015
(86) PCT No.: PCT/US15/50854

§ 371 Date: Mar. 13, 2017

Related U.S. Application Data
(60) Provisional application No. 62/071,285, filed on Sep. 18, 2014.

(51) Int. Cl. A61B 5/042; A61B 5/00

(52) U.S. Cl. CPC A61B 5/0422; A61B 5/6858

(57) ABSTRACT

A basket style electrical mapping catheter includes an elongated body with a proximal end and a distal end, where the proximal end has a user interface for controlling a basket-shaped electrode assembly that extends from the distal end of the elongated body. The basket-shaped electrode assembly includes a plurality of flexible splines supporting measurement electrodes configured to contact an electrically active substrate, and an expander spline disposed along a central axis of the basket-shaped catheter assembly supported a reference electrode. The orientation of the measurement electrodes relative to the reference electrode allows for electrical mapping to be conducted with greater sensitivity and specificity in order to more accurately detected diseased or damaged substrate.

### CROSS-REFERENCE TO RELATED APPLICATIONS

This application claims priority to co-pending U.S. Provisional Patent Application No. 62/071,285 filed on Sep. 18, 2014, the entire content of which is incorporated herein by reference.

### FIELD OF THE INVENTION

The present invention relates to catheters for mapping electrophysiological substrate, and more particularly to basket style catheters for cardiac mapping.

### BACKGROUND OF THE INVENTION

Intravenous electrophysiology catheters are frequently used to identify diseased, or pro-arrhythmic, substrates including infarcted, scarred, or fibrotic cardiac tissues. These catheters typically use closely spaced electrodes to measure the difference in electrical potential between two regions of tissue, known as bipolar electrograms (BPE). Regions of diseased myocardium are known to produce low voltage BPEs. However, accurate detection of diseased tissues with current clinical catheter technologies is limited due to suboptimal orientations of the recording electrodes. Specifically, current clinical systems measure a difference in electrical potential between two recording electrodes that are each in contact with the tissue.

### SUMMARY OF THE INVENTION

The present invention provides, in one aspect, a catheter having an elongated body with a proximal end and a distal end, a basket-shaped electrode assembly extending from the distal end that includes a plurality of flexible splines supporting a plurality of measurement electrodes and an expander spline disposed in the center of the basket-shaped electrode assembly supporting at least one reference electrode.

The present invention provides, in another aspect, a system for mapping an electrophysiological substrate, including a clinical processing unit configured to gather data from a catheter including a basket-shaped electrode assembly that includes a plurality of flexible splines supporting a plurality of measurement electrodes and a expander spline disposed in the center of the basket-shaped electrode assembly supporting at least one reference electrode, where the clinical processing unit gathers data from the measurement electrodes relative to the reference electrode in order to generate a map of electrical conduction within the electrophysiological substrate.

The present invention provides, in another aspect, a method for gathering electrical conductive data from an electrophysiological substrate using a catheter with a basket-shaped electrode assembly having a plurality of flexible splines supporting a plurality of measurement electrodes and a expander spline disposed in the center of the basket-shaped electrode assembly supporting at least one reference electrode, including measuring a difference in electrical potential between the measurement electrodes, which contact the electrophysiological substrate, and the at least one reference electrode, which is spaced from the electrophysiological substrate and disposed within the center of the basket-shaped electrode assembly.

Other features and aspects of the invention will become apparent by consideration of the following detailed description and accompanying drawings.

### BRIEF DESCRIPTION OF THE DRAWINGS

FIG. 1 is a perspective view of a catheter.

FIG. 2 is a schematic diagram illustrating a system for generating an electrical map using the catheter of FIG. 1.

FIG. 3 is a perspective view of a distal end of the catheter of FIG. 1 in a placement position.

FIG. 4 is a perspective view of the distal end of the catheter of FIG. 1 in an operating position.

FIG. 5 is a graph illustrating the correlation between an incidence of angle and specificity/sensitivity of the catheter of FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

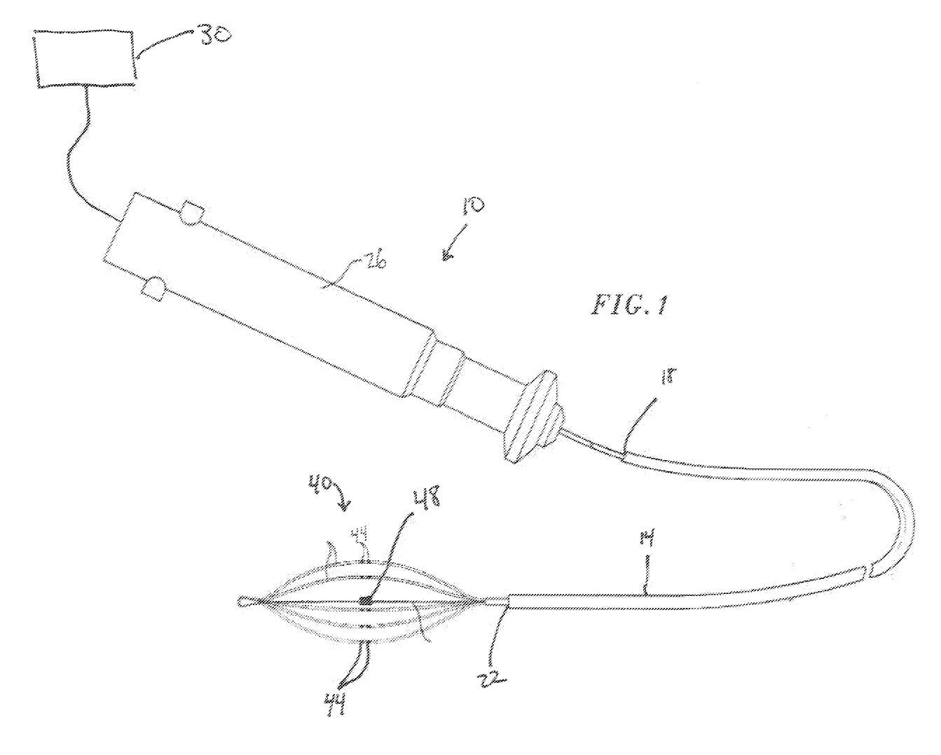

FIGS. 1-5 illustrate basket-shaped catheter 10 and associated system for using the catheter to generate an electrical map according to one embodiment of the invention. The catheter 10 includes an elongated body 14 having a proximal end 18 and a distal end 22, where a user interface 26 and a computational interface 30 extend from the proximal end 18 and a basket-shaped electrode assembly 40 extends from the distal end 22. The basket-shaped electrode assembly 40 includes, among other things, a plurality of measurement electrodes 44 disposed about an outer periphery of the assembly 40 at oblique angles relative to a reference electrode 48 disposed centrally in an interior volume of the basket-shaped electrode assembly 40. In operation, this catheter 10 may be used to, for example, collect electrical data from the interior of chambers of the heart and, in conjunction with a peripheral processing unit or system 34, generate an a map of electrical conduction within the heart. This information may be used clinically to identify infarcted or otherwise diseased cardiac tissue, among other applications.

With reference to FIG. 1, the elongated body 14 forms an insulating barrier and housing member to communicate structural and electrical features of the user interface 26 and the computational interface 30 with the distal end 22 of the elongated body 14. Such members may include electrically-conductive lead wires coupled to the electrode assembly 40, sensor cables, and structural wires or cables for controlling movement of the elongated body 14 and distal end 22, among other members. The elongated body 14 may come in a variety of shapes and sizes to accommodate different types of user interfaces 26 and computational interfaces 30 and associated members for communication with the distal end 22. The elongated body 14 is constructed from a flexible, yet controllable, biocompatible material so as to allow the catheter 10 to be moved effectively through, for example, the cardiac system of a patient for placement of the distal end 22 within the heart without high risk of clinical complication and morbidity. Additionally, the elongated body 14 may be formed of an anti-coagulant material or alternatively may be coated with an anti-coagulant to increase overall biocompatibility.

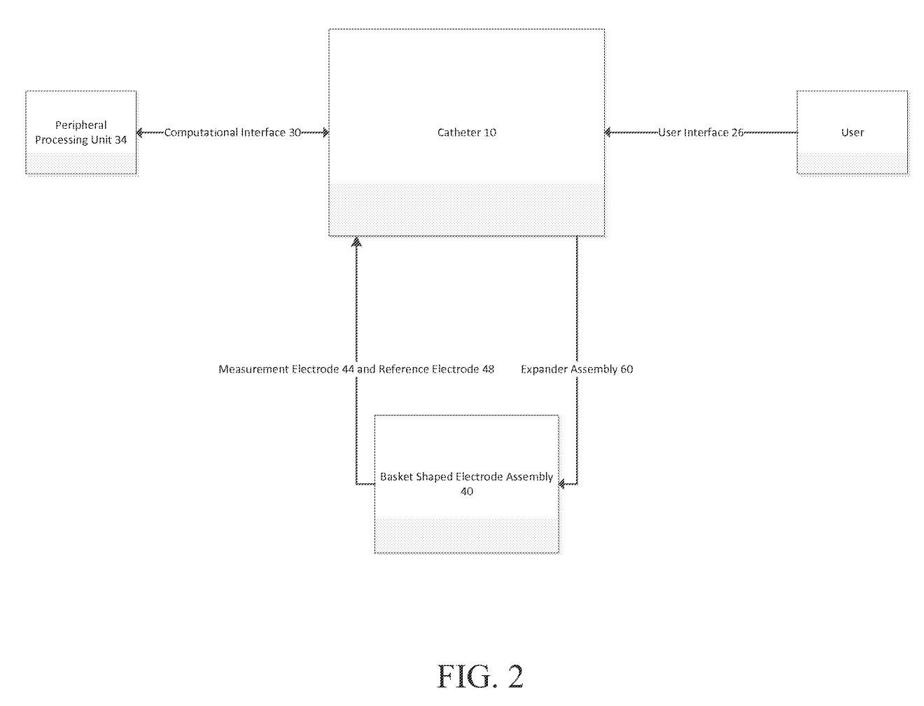

With reference to FIG. 2, the catheter 10 is configured to be used with a peripheral processing unit via the computational interface 30. The computational interface 30 may be any type of mechanical and electrical interface to allow the catheter 10 to communicate data with customized or commercially-available clinical computational, diagnostic, or recording systems 34. In some embodiments, such systems 34 are configured to gather and process electrical data from the catheter 10 to generate clinically-relevant diagnostic data, such as localized regions of decreased electrical conduction in the heart, which may be indicative of a diseased, pro-arrhythmic substrate including infarcted, scarred, or fibrotic cardiac tissue. In other embodiments, the system 34 may be configured to generate a map of electrical conduction of the substrate for various clinical purposes. Although these specific examples have been presented, other clinically-available systems 34 have been considered as useful to this design.

The user interface 26 on the proximal end 18 may be any operating means known the art for controlling the movement of the elongated body 14 and steering the distal end 22 through the body, e.g. blood vessels, as well as for controlling the basket-shaped electrode assembly 40. Examples may include strictly mechanical means which utilize the mechanical conduction of user input to guide the movements of the elongated body 14, distal end 22, and basket-shaped electrode assembly 40. Other examples may include electronically-controlled systems in which a user operates the catheter 10 via a computer interface.

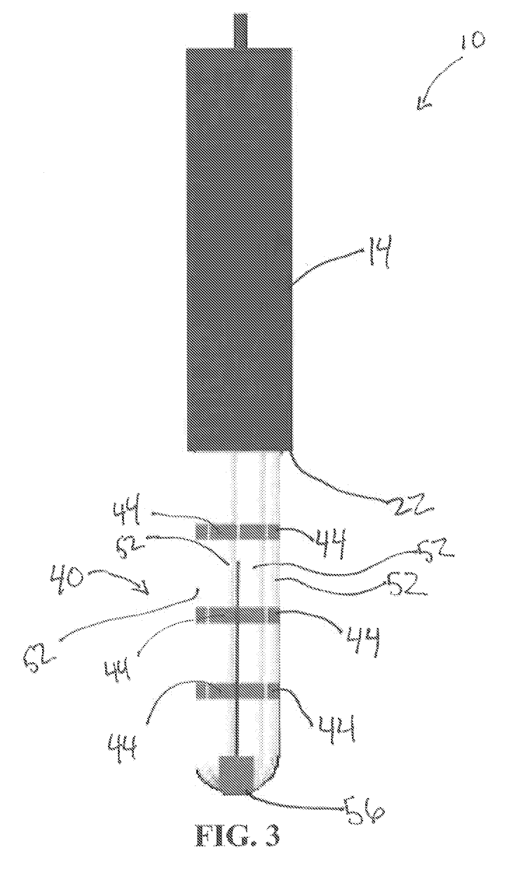

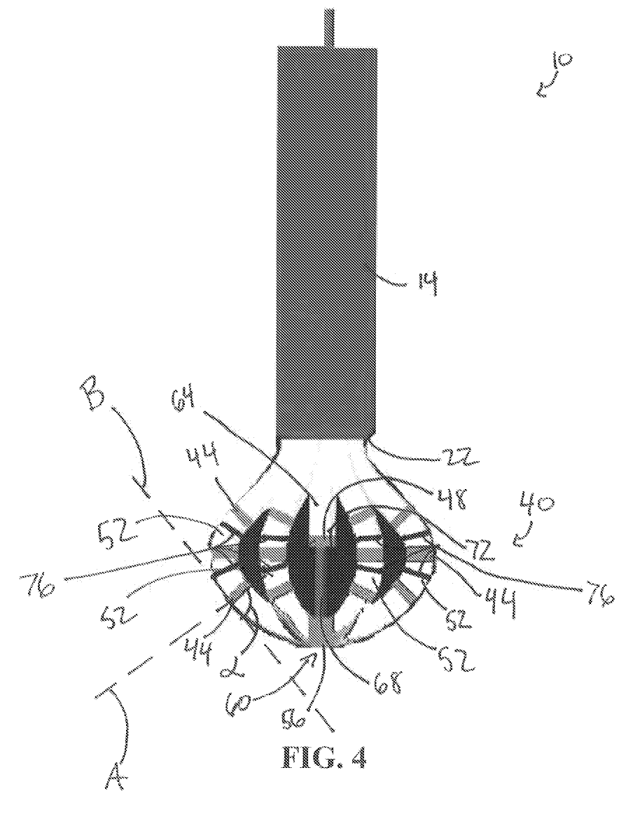

With reference to FIGS. 3-4, the distal end 22 of the elongated body 14 and the basket-shaped electrode assembly 40 are shown. The basket-shaped electrode assembly 40 includes a plurality of flexible splines 52 (e.g., generally three or more splines) supporting a plurality of measurement electrodes 44, extending longitudinally away from the distal end 22 of the elongated body 14, and meeting at a common coupling end 56. The flexible splines 52, together with the coupling end 56, define an outer periphery of the basket assembly 40 that is generally coaxial with the distal end 22 of the elongated body 14. When the catheter 10 is deployed, the space within the flexible splines 52 and the coupling end 56 define an inner volume. An expander assembly 60, including a central spline 64 supporting at least one reference electrode 48 and a retractable cable 68, extends substantially through the center of the inner volume and connects to the coupling end 56. The central spline 64 extends from the distal end 22 of the elongated body 14 to approximately 25%-75% of the length of each flexible spline 52, and includes a bore 72 supporting the retractable cable 68. As seen most clearly in FIG. 4, the retractable cable 68 extends from the bore 72 of the central spline 64 to the coupling end 56 of the basket-shaped electrode assembly 40. The cable 68 is operatively coupled to the user interface 26 through the bore 72 and elongated body 14, and has a length that may be adjusted by a user via the user interface 26 in order to operate the basket-shaped electrode assembly 40 between a placement position (FIG. 3) and an operating or deployed position (FIG. 4).

The plurality of measurement electrodes 44, disposed on the flexible splines 52, and at least one reference electrode 48, disposed on the central spline 64, are configured to measure a difference in electrical potential. In some embodiments, each flexible spline 52 may include 1 to 10 measurement electrodes 44, and preferably about four measurement electrodes 44, that are spaced along the splines at regular intervals. However, other configurations and spacing of the measurement electrodes 44 may be used.

As illustrated in FIG. 4, the central spline 64 supports the reference electrode 48 near a distal end of the central spline 64. However, depending on the overall length of the central spline 64 relative to the flexible splines 52, the reference electrode 48 may be disposed between approximately the distal end of the central spline 64 and approximately half of the length of the central spline 64. Preferably, in any configuration of the central spline 64, the reference electrode 48 should be disposed approximately in the center of the inner volume when the basket-shaped electrode assembly 40 is in the operating position. With continued reference to FIGS. 3 and 4, the expander assembly 60 is configured to operate the basket-shaped electrode assembly 40 between a placement position (FIG. 3) and an operating position (FIG. 4). In the placement position, the retractable cable 68 is fully extended so the entire expander assembly 60 is substantially the same length as the flexible splines 52, thereby allowing the flexible splines 52 to extend substantially parallel to the outer periphery of the distal end 22 of the elongated body 14. This position allows a user to more easily move the catheter 10 within the patient prior to collecting any data with the catheter 10.

Once the user has operated the catheter 10 into the desired location (e.g., chamber of the heart), the user interface 26 may be operated to displace the coupling end 56 toward the distal end 22 of the elongated body 14 by adjusting the length of the retractable cable 68. This displacement, combined with the flexibility of the flexible splines 52, allows the splines 52 to bend so as to curve radially outwardly, thereby causing the basket-shaped electrode assembly 40 to take on a substantially three-dimensional (e.g. spherical) shape. In other embodiments, the flexible splines 52 may bend or curve to form other three dimensional shapes, such as an ovoid ‘egg’ shape or other, possibly irregular, shapes. In addition, the flexibility of the splines allows the flexible splines 52 to adjust to the contour of a wide variety of surfaces with which the splines 52 come into contact when the basket assembly 40 is moved into the operating or deployed position.

As seen in FIG. 4, the shape of the flexible splines 52 relative to the expander assembly 60, and the ability of the splines to match the contour of any surface which the splines contact, allows a line A extending through the measurement electrodes 44 and reference electrode 48 to maintain an oblique angle α relative to a line B tangent to the point of contact of the measurement electrode 44 (i.e., a plane defined by the surface that the spline contacts). In some embodiments, the angle α may be between approximately 15°-90° and, more specifically, between about 30°-75°.

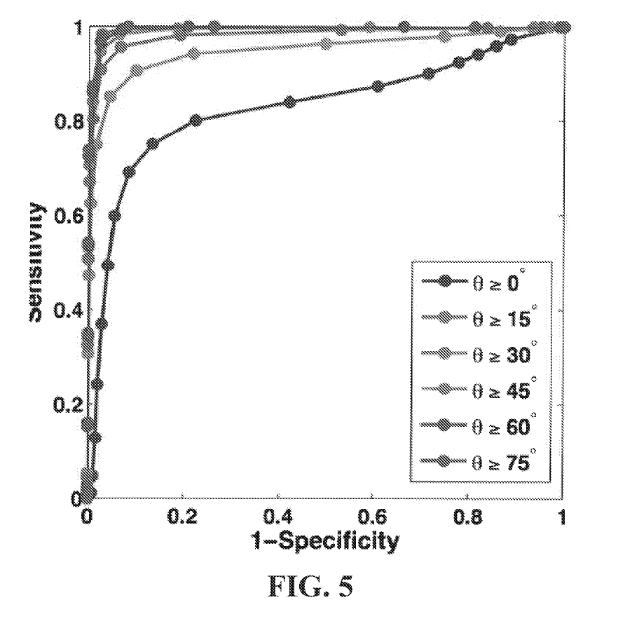

The relationship between the angle a and measurement sensitivity as well as specificity is seen in FIG. 5. The oblique angle α between the measurement electrodes 44 and the reference electrode 48, specifically when the angle α is greater than 15°, substantially increases the sensitivity and specificity of electrical data collected by the catheter 10. This allows a clinician to more easily identify diseased or damaged tissue and/or diagnose conditions involving improper electrical conduction within the heart.

As illustrated in FIG. 4, the measurement electrodes 44 are disposed at a distance d relative to the reference electrode 48 in the operating position. The distance d between the reference and measurement electrodes 44 should be greater than a first length, in order to realize a large enough difference in the signal obtained from the reference electrode 48 and the signal obtained from measurement electrode 44. Furthermore, the distance cannot be larger than a second length as it increases the difference between common noise measured at each electrode, thereby reducing the accuracy of the signal. For example, if the basket-shaped electrode assembly 40 is deployed within the atria, the measurement electrode 44 and reference electrode 48 may receive electrical signals from the ventricles (i.e., noise). If the electrodes are spaced at a distance larger than the second length, the noise will not be received at the same time and at the same amplitude at each electrode, meaning the noise will not be effectively attenuated during computation. The distance d, in the operating position, is between about 0.2 cm and 5 cm, preferably about 0.5 to 1 cm.

In another embodiment, the basket-shaped electrode assembly 40 may include other sensors 76 or means for determining when the flexible splines 52 and/or measurement electrodes 44 come into contact with the substrate, such as force or temperature sensors 76. These sensors 76 may be integrated with the electrodes 44 or may be located on the splines 52 between the electrodes 44 or co-localized with the electrodes 44. Alternatively, the electrodes 44 themselves may be configured to determine if the electrodes 44 are in contact with the substrate. This allows the user to determine if the measurements being obtained from each measurement electrode 44 relative to the reference electrode 48 are erroneous due to a lack of contact with the substrate or other reasons. Furthermore, these sensors 76 or other such means may be operable to localize the measurement electrodes 44 relative to the reference electrode 48 to, for example, confirm the angle α is sufficient for a quality reading. In one construction, this may be accomplished by measuring impedance between the electrodes, although other techniques and sensors may also be used.

Various features of the invention are set forth in the following claims.

What is claimed is:

1. A catheter, comprising:
an elongated body having a proximal end and a distal end;
a basket-shaped electrode assembly extending from the distal end of the elongated body, where the basket-shaped electrode assembly includes a plurality of flexible splines defining an interior volume and at least one central spline disposed within the interior volume, where each of the flexible splines and the central spline are coupled at an end spaced from the distal end of the elongated body;
a plurality of measurement electrodes disposed on the plurality of flexible splines; and
at least one reference electrode disposed on the central spline within the interior volume.

2. The catheter of claim 1, wherein the basket-shaped electrode assembly is movable between a placement position in which the basket-shaped electrode assembly has a cylindrical shape and an operating position in which the basket-shaped electrode assembly has a spherical shape.

3. The catheter of claim 2, wherein, in the operating position, the at least one reference electrode is disposed near the center of the spherical shape defined by the basket-shaped electrode assembly.

4. The catheter of claim 2, wherein, in the operating position, at least one measurement electrode contacts a substrate at a surface and forms an oblique angle between the measurement electrode and the at least one reference electrode relative to an axis orthogonal to the tissue.

5. The catheter of claim 4, wherein the angle is between approximately 15 degrees and 90 degrees.

6. The catheter of claim 1, wherein the basket-shaped electrode assembly is configured to be deployed within a chamber of a patient's heart, and the measurement electrodes are configured to contact a wall of the chamber while the reference electrode is configured to remain spaced at a distance from the wall of the chamber.

7. A system for mapping an electrophysiological substrate, comprising:
a clinical processing unit configured to gather data from a catheter, the catheter including a basket-shaped electrode assembly that includes a plurality of flexible splines supporting a plurality of measurement electrodes and a expander spline disposed in the center of the basket-shaped electrode assembly supporting at least one reference electrode;
wherein the clinical processing unit gathers data from the measurement electrodes relative to the reference electrode in order to generate diagnostic data about the electrophysiological substrate.

8. The system of claim 7, wherein the basket-shaped electrode assembly is movable between a placement position in which the basket-shaped electrode assembly has a substantially cylindrical shape and an operating position in which the basket-shaped electrode assembly has a substantially spherical shape.

9. The system of claim 8, wherein, in the operating position, at least one measurement electrode contacts a substrate at a surface and form an oblique angle between the measurement electrode and the at least one reference electrode relative to an axis orthogonal to the tissue.

10. The system of claim 8, wherein, in the operating position, the measurement electrodes are in contact with the electrophysiological substrate and the at least one reference electrode is spaced from the electrophysiological substrate.

11. A method for gathering electrical conductive data from an electrophysiological substrate using a catheter with a basket-shaped electrode assembly having a plurality of flexible splines supporting a plurality of measurement electrodes and a expander spline disposed in the center of the basket-shaped electrode assembly supporting at least one reference electrode comprising:
measuring a difference in electrical potential between the measurement electrodes, which contact the electrophysiological substrate, and the at least one reference electrode, which is spaced from the electrophysiological substrate and disposed within the center of the basket-shaped electrode assembly.

12. The method of claim 11, further including, prior to the step of measuring, inserting the catheter intravenously into a patient, directing the basket-shaped electrode assembly into a chamber of the patients heart, and moving the basket-shaped electrode assembly from a cylindrical placement position into a spherical operating position.

13. The method of claim 11, wherein the reference electrode does not contact the electrophysiological substrate during measuring.

14. The method of claim 11, wherein the measurement electrodes are disposed concentrically about the at least one reference electrode.

15. A basket-shaped electrode assembly for a catheter, comprising:
a plurality of flexible splines and at least one expander spline disposed within the flexible splines;
a plurality of measurement electrodes disposed on the plurality of flexible splines; and
at least one reference electrode disposed on the expander spline;
wherein the measurement electrodes are disposed concentrically about the at least one reference electrode.

16. The basket-shaped electrode assembly of claim 15, wherein the plurality of flexible splines are movable between a placement position in which the plurality of flexible splines have a cylindrical shape and an operating position in which the plurality of flexible splines have a spherical shape.

17. The basket-shaped electrode assembly of claim 16, wherein, in the operating position, the at least one reference electrode is disposed near the center of the spherical shape defined by the plurality of flexible splines.

18. The basket-shaped electrode assembly of claim 16, wherein, in the operating position, at least one measurement electrode contacts a substrate at a surface and forms an oblique angle between the measurement electrode and the at least one reference electrode relative to an axis orthogonal to the tissue.

19. The basket-shaped electrode assembly of claim 18, wherein the angle is between approximately 15 degrees and 90 degrees.
